(19) United States
(12) Patent Application Publication
(10) Pub. No.: US 2009/0075835 A1
(43) Pub. Date: Mar. 19, 2009

(54) METHODS AND SYSTEMS FOR THE DETECTION OF MICRODELETION AND MICRODUPLICATION SYNDROMES

(75) Inventors: Bassem A. Bejjani, Spokane, WA (US); Blake Charles Ballif, Greenacres, WA (US); Lisa G. Shaffer, Spokane, WA (US)

Correspondence Address: SPECKMAN LAW GROUP PLLC, 1201 THIRD AVENUE, SUITE 330, SEATTLE, WA, 98101, US

(73) Assignee: SIGNATURE GENOMICS LABORATORIES, LLC, Spokane, WA (US)

(21) Appl. No.: 12/211,578

(22) Filed: Sep. 16, 2008

Related U.S. Application Data
(60) Provisional application No. 60/973,141, filed on Sep. 17, 2007.

(51) Int. Cl. C40B 30/04; C40B 40/08; C12Q 1/68

(52) U.S. Cl. 506/9; 506/17; 435/6

(57) ABSTRACT

Methods for diagnosing the presence or absence of a genetic disorder in a patient are provided, wherein the genetic disorder is associated with a chromosomal abnormality at 1q41q42 and/or 16p11.2p12.2, and wherein the genetic disorder is not Fryns syndrome or congenital diaphragmatic hernia (CDH). Materials, such as microarrays for use in microarray CGH, and kits for use in such methods are also provided.

### REFERENCE TO RELATED APPLICATIONS

This application claims priority to U.S. provisional patent application No. 60/973,141, filed Sep. 17, 2007.

### FIELD OF THE INVENTION

The present invention relates generally to the diagnosis of disorders and more specifically to the diagnosis of syndromes associated with specific DNA copy number changes.

### BACKGROUND OF THE INVENTION

Microdeletion syndromes are a heterogeneous group of genetic disorders caused by the deletion of specific small regions of chromosomal DNA. These deletions are difficult to visualize using standard cytogenetic techniques. In the past, identification of chromosomal abnormalities has depended on the identification of a specific phenotype in one or more patients followed by detection of the associated genotype, generally by identification of a chromosomal rearrangement visible by Giemsa-banding (G-banding). For example, banded chromosomes have been important in the identification of the chromosomal basis in syndromes such as Prader-Willi syndrome, Williams-Beuren syndrome and DiGeorge syndrome. However, G-banding cannot reliably identify abnormalities of less than five Mb.

In 1995, Flint et al. (Nat Genet 1995;9:132-140) developed a method for simultaneously interrogating all the unique human subtelomeres using fluorescence in situ hybridization (FISH). The use of subtelomere FISH panels has illustrated that, in the absence of specific clinical features suggestive of a syndrome, patients with mental retardation can be “screened” for a novel chromosomal abnormality. This development represented a shift from the traditional “phenotype-first” approach explained earlier, wherein a set of patients was grouped based upon shared clinical features, to a “genotype-first” approach by which individuals can be characterized first by a common cytogenetic aberration and then, as more patients with the same abnormality are ascertained, a clinical presentation can be developed.

The development of comparative genomic hybridization (CGH), particularly CGH using microarrays (array CGH), broadened the scope and resolution at which the genotypes of patients with idiopathic mental retardation could be assayed. CGH provides genome-wide screening of genetic sequence alterations by comparing differentially labeled test and control samples of genomic DNA. In contrast to subtelomere FISH, microarrays for use in CGH may be constructed with contiguous or non-contiguous coverage of the entire genome or with consideration of well-known microdeletion and microduplication syndrome loci; thus, the principles of subtelomere FISH panels may be applied to a much larger proportion of the genome. For example, in screening patients with unexplained overgrowth syndrome by whole-genome array CGH, Redon et al. (Eur J Hum Genet 2006;14:759-767) identified two individuals with de novo interstitial deletions of 9q22.32-q22.23. Although the breakpoints differed in each patient, the similarity of clinical features—both individuals presented with macrocephaly, overgrowth, trigonocephaly and hyperactivity in addition to a constellation of distinct facial features—led the authors to suggest that deletion of 9q22.3 was a novel microdeletion syndrome.

While many syndromes associated with specific microdeletions and microduplications have been identified, it is likely that many others remain unidentified. The inventors have taken a directed, functional approach to identify individuals with novel microdeletion syndromes and their reciprocal microduplications.

### SUMMARY OF THE INVENTION

The present invention provides methods and systems for the detection of one or more genetic disorders, such as microdeletion and microduplication syndromes, involving cytogenetic abnormalities of chromosomal loci of interest. Such cytogenetic abnormalities involve abnormal copy numbers of nucleic acid sequences such as, but not limited to, amplifications (for example duplications) and/or deletions of sequences. In specific embodiments, the chromosomal loci of interest are 1q41q42 and 16p11.2p12.2.

In one aspect, methods are provided that comprise: (a) providing a DNA-containing test sample from a patient, and (b) identifying the deletion or amplification of target chromosome loci in the test sample, wherein the target chromosome loci are selected from the group consisting of: 1q41a42 and 16p 1.2p12.2, wherein the absence or amplification of the target chromosome loci is indicative of the presence of a genetic disorder in the patient and wherein the genetic disorder is not Fryns syndrome or congenital diaphragmatic hernia (CDH).

In one such embodiment, the genetic disorder that may be detected using the disclosed methods is a microdeletion syndrome that correlates with a deletion of 1q41q42, and is characterized by the following phenotype: significant developmental delay and distinct facial dysmorphism (frontal bossing, deep-set eyes, broad nasal tip, depressed nasal bridge, anteverted nares), coarse facies in infancy, microcephaly, cleft palate, clubfeet, seizures, short stature, diaphragmatic hernia and lung hypoplasia, and possibly having the clinical diagnosis of Fryns syndrome.

In a second embodiment, the genetic disorder is a microdeletion syndrome that correlates with a deletion of 16p11.2p12.2 and is characterized by the following phenotype: developmental delay and distinct facial dysmorphism; Pierre Robin Sequence (cleft lip and palate, glossoptosis, micrognathia); downslanting palpebral fissures; bilateral epicanthal folds; deep-set eyes; absent tear ducts; strabismus; low-set and malformed ears; short, prominent nose; and small stature.

In yet a further embodiment, the genetic disorder correlates with an amplification, such as a duplication or triplication, of 16p11.2p12.2.

Techniques for the detection of abnormal copy numbers in chromosomal loci are well known in the art, and include, for example, microarray comparative genomic hybridization (CGH), SNP microarray, fluorescence in situ hybridization (FISH), restriction fragment length polymorphism (RFLP), polymerase chain reaction (PCR), Southern blotting, pulse field gel electrophoresis (PFGE), multiplex ligation dependent probe amplification (MLPA), and real time PCR.

In one embodiment, the presence or absence of a chromosomal abnormality in a patient is determined by means of microarray CGH and the method includes the following steps: (a) providing a DNA-containing sample from the patient; (b) labeling nucleic acids from the test sample with a first detectable label; (c) labeling nucleic acids from a control sample with a second, different, detectable label; (d) contacting the labeled nucleic acids from each sample with a plurality of target nucleic acids specific for chromosomal loci selected from the group consisting of 1q41a42 and 16p11.2p12.2; and (e) comparing the intensities of the signals from labeled nucleic acids hybridized to each target nucleic acid, thereby allowing detection of the presence or absence of the chromosomal abnormality in the test sample.

In other aspects materials for the detection of chromosomal abnormalities are provided. Such materials include microarrays comprising at least one target nucleic acid specific for chromosomal loci selected from the group consisting of 1q41q42 and 16p11.2p12.2. Kits for use in the disclosed methods are also provided, such kits comprising a container and at least one nucleic acid probe or primer pair that is capable of detecting the presence or absence of a chromosomal abnormality, such as a deletion or amplification, at 1q41q42 and 16p11.2p12.2. Nucleic acid probes that may be effectively employed in such kits are capable of specifically hybridizing to a region of 1q41q42 or 16p11.2p12.2. Primer pairs that may be effectively employed in such kits are capable of specifically amplifying a region of 1q41q42 or 16p11.2p12.2.

These and additional features of the present invention and the manner of obtaining them will become apparent, and the invention will be best understood, by reference to the following more detailed description and the accompanying drawings.

### BRIEF DESCRIPTION OF THE DRAWINGS

The present invention will be described in greater detail in the following detailed description, with reference to the accompanying drawings, wherein:

FIG. 1 shows results for chromosome 1 obtained using a microarray. Each clone on the plot is arranged along the x-axis according to its location on the chromosome with the most distal 1p telomeric clones on the left and the most distal/telomeric 1q clones on the right. The line indicated by ♦ represents the control:subject fluorescence intensity ratios for each clone, whereas the line indicated by ▪ represents the fluorescence intensity ratios obtained from a second hybridization in which the dyes have been reversed (subject:control). (A) Normal plot for chromosome 1. Note that all data points are at a log 2 ratio of zero. (B) Plot for subject 2 showing a deletion from BAC RP11-1031M6 through BAC RP11-61M2. (C) Plot for subject 3 showing a deletion from BAC RP11-1148E24 through BAC RP11-61M2. (D) Plot for subject 1 showing a deletion from BAC RP11-208F18 through RP11-61M2. For each of the plots B, C, and D, the deletion is identified as a mirror image deviation from a log 2 ratio of zero to a log 2 ratio of ˜0.3 to −0.3.

FIG. 2 shows results for chromosome 1 obtained using a high-density oligonucleotide microarray. Copy number data for a selected region of chromosome 1 (210,000,000-230,000,000, NCBI build 35) is shown for (A) subject 1, (B) subject 2 and (C) subject 3. The plots show the signal intensity ratio (10g2 ratio) of each probe on the Affymetrix 250K Sty chips resulting from analysis with CNAG software (Nannya Y, et al. Cancer Res 2005;65:6071-6079). The dots represent raw log₂ ratio values for each SNP. The curves represent copy number inferences based on local mean analysis for five consecutive SNPs. Heterozygous SNP calls are shown as non-bold bars below the chromosome. For probes that are normal copy number, the signal intensity ratio of the subject vs. controls is expected to be 1 and log₂ ratio should be around 0.0 (log₂1=0). The deletions detected in subjects 1-3 based on log₂ ratio are underlined in bold. Loss of copy number due to deletion in the subjects results in a negative log₂ ratio (mean log₂ ratio ˜−0.5).

FIG. 3 shows breakpoint locations for subjects with deletions of 1q41q42. The chromosome bands of the 1q42 region are shown with the distance from the 1p telomere in Mb along the top (based on the University of California Santa Cruz May 2004 draft of the human genome). The horizontal bars indicate the deleted regions for each patient based on the array CGH data of Table 1, with the gray bars for subject 7 indicating the regions containing the breakpoints based on the data in the publication by Kantarci et al. (Am J Med Genet A 2006;140:17-23). The vertical bar indicates the smallest region of overlap (SRO) defined by the breakpoints in these subjects and contains the DISP1 locus.

FIG. 4A-E shows an analysis of individuals with copy-number imbalances of 16p11.2p12.2. (A) Pericentromeric array CGH profile of a >4.99-Mb deletion of 16p11.2p12.2 in case 1. For the array CGH plots, clones are ordered on the x-axis according to physical mapping positions. The top plot shows a normal chromosome 16; the bottom plot shows the abnormal chromosome 16. (B) Pericentromeric array CGH profile of a de novo triplication of ˜4 Mb of 16p11.2p12.1 and duplication of ˜1 Mb of 16p12.1p12.2 in case 5. Microarray plots are arranged as in FIG. 1A. (C) NimbleGen™ whole-genome oligonucleotide array CGH profiles for cases 1, 2, 4 and 5. (D) Affymetrix 250K SNP array profile for case 3. (E) Schematic of the 16p11.2p12.2 region with a summary of the abnormalities identified in five patients. A simplified interpretation of the segmental duplications located in 16p11.2p12.2 are shown with their relative orientations (arrows). The block of segmental duplications between C5 and A4 does not share identity with the 16p11.2p12.2 region. Some of the RP11 BAC clones used for FISH confirmation of abnormalities are shown as dots along the chromosome. Horizontal bars for cases 1-4 indicate deleted regions for each patient. The left-hand and right-hand bars for case 5 indicate regions of duplication and triplication, respectively. Regions of copy number variation based on the Database of Genomic Variants are indicated at the bottom of the figure. The locations of select genes from >100 known genes in the region are shown.

FIG. 5 shows genomic coordinates of BAC clones used in pericentromeric array CGH and FISH to delineate breakpoints in patients with abnormalities of 16p11.2p12.2, based on the University of California Santa Cruz May 2004 draft of the human genome.

### DETAILED DESCRIPTION OF THE INVENTION

As outlined above, methods and materials for identifying the presence or absence of a genetic disorder in a patient, such as a microdeletion syndrome, associated with a chromosomal abnormality at 1q41q42 and/or 16p11.2p12.2 are provided. As detailed below in the Examples section, the inventors have identified novel microdeletion syndromes associated with deletions of 1q41q42 and/or 16p11.2p12.2, together with a novel genetic disorder associated with amplification (such as duplication or triplication) of 16p11.2p12.2.

As used herein, the term “patient” refers to a mammal, such as a human. The methods described herein involve providing a DNA-containing sample from a patient suspected of having a genetic disorder. Examples of DNA-containing samples that may be used in the methods include, but are not limited to, blood, serum, urine, saliva, amniotic fluid, chorionic villus, semen, skin, or any tissue or bodily fluid from which DNA can be extracted.

Techniques that may be used to identify the presence or absence of chromosomal abnormalities in DNA-containing samples are well known in the art and include, for example, microarray CGH, FISH, PCR, real-time PCR, PFGE, MLPA, RFLP and Southern blotting.

Many of these techniques employ nucleic acid probes that are capable of specifically hybridizing to target chromosomal loci, such as 1q41a42 or 16p11.2p12.2. Such probes are at least substantially complementary to unique sequences in the target chromosomal loci. As used herein, the term “substantially” complementary is used to describe the commonly understood interaction of complementary base pairing. Imperfect pairing, whether due to deletions or imperfect base matching, is envisioned provided that the pairing results in hybridization. In certain embodiments, the probes are fully complementary to a region of the target chromosome loci.

Hybridization conditions suitable for use in the disclosed methods are well known in the art. In general, hybridization conditions include temperatures of about 25° C. to about 55° C., and incubation lengths of about 0.5 hours to about 96 hours. Non-specific binding of probes to DNA outside of the target chromosome loci can be removed by a series of washes. The temperature and concentration of salt in each wash will depend on the desired stringency. For example, for high stringency conditions, washes can be carried out at about 65° C. to about 80° C. using 0.2× to about 2×SSC, and about 0.1 % to about 1% of a non-ionic detergent such as Nonidet P-40. Stringency can be lowered by decreasing the temperature of the washes or by increasing the concentration of the salt in the washes.

Probes for use in the disclosed methods are typically about 20 to about 5×10⁵ nucleotides in length. Suitable probes may be obtained commercially, for example from Vysis, Inc. (Downers Grove, Ill.), Molecular Probes, Inc. (Eugene, Oreg.) or from Cytocell Technologies, Inc. (Oxfordshire, UK), or can be prepared from chromosomal or genomic DNA using standard techniques, such as that described in U.S. Pat. No. 6,303,294, the disclosure of which is hereby incorporated by reference. For example, probes can be obtained by preparing primer pairs that are effective to amplify a region shown to be unique in the target chromosome loci, synthesizing DNA that is substantially complementary to a region of normal human genomic DNA or cDNA by PCR amplification using the primer pairs, and isolating probes for the target chromosome locus.

For use in the disclosed methods, the probes are generally labeled with one or more detectable labels, such as radioisotopes, fluorescers, chemiluminescers, stains, enzymes and antibodies. Labeled nucleic acid probes for use in microarray CGH can be prepared as taught, for example, in U.S. Pat. No. 7,011,949, the disclosure of which is hereby incorporated by reference. Copy number gains and losses can be detected using probes within the genomic interval 21,261,364-30,262,280 on human chromosome 16 (based on the NCBI Build 35, May 2004 Assembly (hg17) of the human genome).

Microarray CGH provides information on the copy number of specific chromosomal loci by comparing the intensities of hybridization signals among different locations on a reference genome. Microarray CGH may be employed to detect abnormal copy numbers of 1q41a42 or 16p11.2p12.2 as described. The design and construction of microarrays for use in CGH, together with techniques for their use, are well known in the art and include, for example, those described in US Patent Application Publication US2005/0181410A1, the disclosure of which is hereby incorporated by reference.

FISH involves the steps of preparing interphase or metaphase spreads, for example from cells of peripheral blood lymphocytes, followed by hybridizing labeled nucleic acid probes to the interphase or metaphase spreads. Probes can be labeled, for example, with biotin-dUTP or digoxigenin-dUTP. Using probes with mixed labels allow visualization of space, order and distance between hybridization sites. FISH can be performed using techniques well known to those of skill in the art, as described for example in U.S. Pat. Nos. 6,562,565 and 5,759,781, the disclosures of which are hereby incorporated by reference.

Chromosomal abnormalities can also be detected by extracting DNA from a test sample and performing Southern blotting analyses using restriction enzyme digest and nucleic acid probes which are capable of hybridizing to a sequence within the target chromosome loci and thereby detecting the presence or absence of the chromosomal abnormality. Dosage measurements from Southern blotting can be obtained, for example, by visual examination, densitometry measurement, quantitative radioactivity and/or quantitative fluorescence.

Alternatively, the presence or absence of chromosomal abnormalities can be determined by extracting DNA from a test sample, amplifying the extracted DNA and performing PCR using primer sequences located within the target chromosome loci. PCR techniques suitable for use in the methods disclosed herein are well known in the art (see, for example, Erlich ed., PCR Technology, Stockton Press, NY, 1989).

### EXAMPLE 1

### Identification of a Novel Microdeletion Syndrome of 1q41q42

### Microarray Hybridization

Blocking of the slides was achieved with 10% bovine serum albumin fraction V (Sigma, St. Louis, Mo., USA) and 20 μg salmon sperm DNA (Invitrogen, Carlsbad, Calif., USA) in a humid chamber at 45° C. for 2 hours. Slides were denatured in boiling millipore water, then dehydrated with ice-cold 95% ethanol and stored in a dessicator. Genomic DNA was extracted (Puregene DNA isolation Kit, Gentra Systems, Inc. Minneapolis, Minn., USA) from lymphoblastoid cell lines, peripheral blood, or cultured tissues of the subjects and phenotypically normal male and female references. Genomic DNA was digested with Dpn II (New England Biolabs, Inc., Beverly, Mass., USA) and reprecipitated (1:8 volume of NaAc 3M pH5.2 and 1:1 volume of isopropanol). A dye-reversal strategy was used on two separate microarrays in which 500 ng of both subject and reference DNAs were labeled (Bio Prime DNA labeling System, Invitrogen) with Cyanine3 (Cy3) or Cyanine5 (Cy5), respectively (Hodgson et al. 2001). The subject and reference DNA were co-hybridized to one microarray and then oppositely labeled and co-hybridized to a second microarray (Wessendorf et al. 2002). Shortly after the labeling, probes were purified with Microcon filter units (Millipore, Billerica, Mass., USA), and ˜500 ng of subjects' DNA, combined with an equal amount of reverse-sex control DNA, was co-precipitated with 50 μg of Cot1-DNA (Invitrogen) and hydrated with 15.5 μl ULTRAhyb (Ambion, Austin, Tex., USA). The labeled genomic DNAs were denatured at 72° C. for 5 minutes, preannealed immediately after at 37° C. for 1 hour, placed onto a microarray, and covered with a 22×22 mm coverslip. Hybridization was performed in an incubation chamber (Coming Incorporated Life Sciences, Acton, Mass., USA) at 37° C. with shaking for 14-16 hours. Following the hybridization, the coverslips were removed in 1×PBS and the microarrays were washed with 50% formamide, 2×SSC, 0.1% SDS at 45° C. for 20 minutes and 1×PBS for 20 minutes at room temperature in the dark. The microarrays were then rinsed with 0.2×SSC and millipore water and dried immediately.

### Microarray Analysis

Images were acquired using a GenePix 4000B (Axon Instruments, Union City, Calif., USA) dual-laser scanner and individual spots were analyzed with GenePix Pro 5.0 imaging software (Axon Instruments). Two simultaneous scans of each array were obtained at wavelengths of 635 nm and 532 nm. The average ratios of the four spots for each subject were analyzed with Acuity 3.1 software (Axon Instruments). After subtraction of background noise, the ratio of fluorescence intensities derived from hybridized test and control DNA was calculated and normalized by the ratios measured from reference spots on the same slide. The average ratios of the four spots for each subject were then converted to a log₂ scale and plotted in Microsoft Excel. We set our threshold for copy-number gain and loss at 0.3 and −0.3, respectively.

### FISH Analysis

FISH was performed as previously described (Shaffer et al., Am. J. Hum. Genet. 55:968-974 (1994)). DNA was extracted from BAC clones using a standard alkaline lysis protocol and labeled by nick translation (Roche Diagnostic, Indianapolis, Ind., USA) with biotin-dUTP or digoxigenin-dUTP (Roche). The signals were amplified with FITC-avidin (Sigma) and anti-avidin (Sigma) to detect the biotin-dUTP, and with anti-digoxigenin monoclonal antibody, anti-mouse IgG-digoxigenin and anti-digoxigenin-rhodamine FAB fragments (Sigma) to detect the dig-dUTP. The slides were counterstained with DAPI. Cells were examined with a Zeiss Axioplan II fluorescence microscope equipped with a triple-bandpass filter that allows multiple colors to be visualized simultaneously. Digital images were captured and stored with Isis software V 3.4.0 (Metasystems, Altlussheim, Germany).

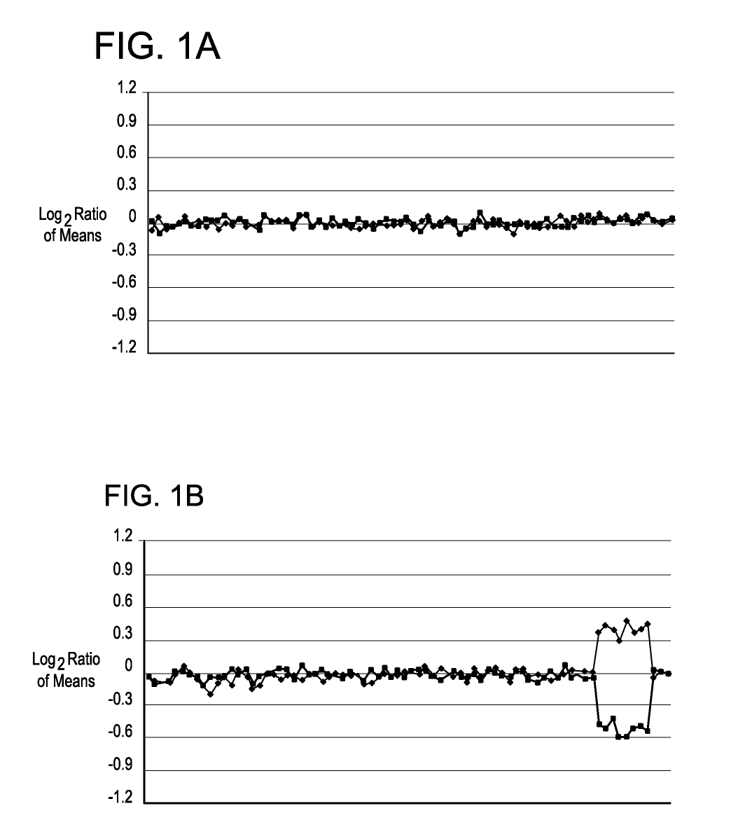

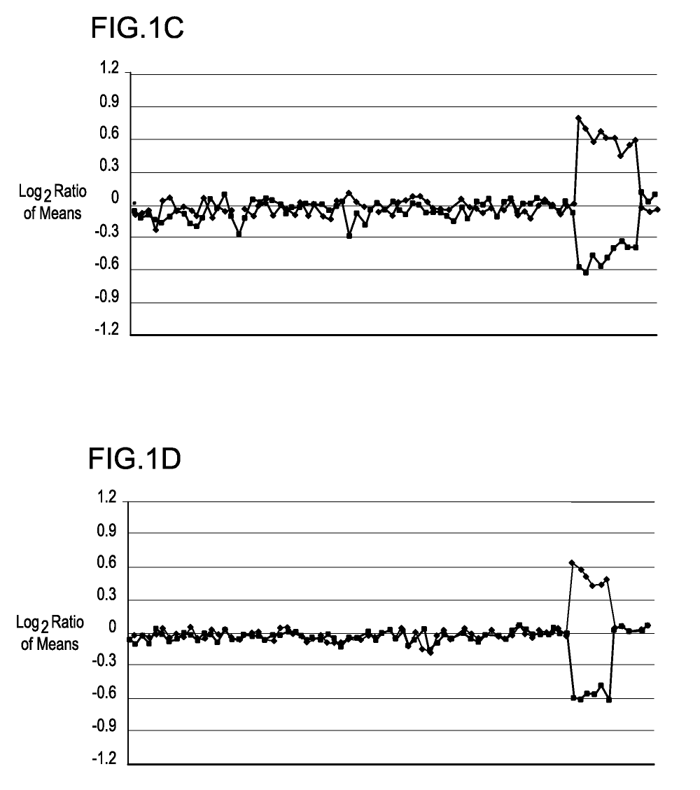

Over 10,000 peripheral blood samples were analyzed by array CGH using the SignatureChip® diagnostic microarray as described above. The targeted microarray used with CGH included regions commonly rearranged in chromosome abnormalities, including microdeletion syndromes and the subtelomeric regions. In addition, to increase coverage over the genome in a deliberate manner, genes active in important developmental pathways were also included, such as PTCH, ZIC2 and DISP1, which are involved in the sonic hedgehog (SHH) signaling pathway, the disruption of which has been implicated in holoprosencephaly (HPE). Furthermore, the pericentromeric areas of all the chromosomes were also covered with BACs that mapped to the most proximal unique regions of the chromosomes. Seven patients were identified with microdeletions of 1q41q42 which includes the DISP1 locus (FIG. 1; Table 1). These patients were subsequently determined to have common clinical features.

TABLE 1
Results for subjects with deletions of 1q41q42.

| | Deletion identified by | Deletion defined by |
|---|---|---|
| Subject | SignatureChip ® analysis | Affymetrix 250K chip^{a} |
| 1 | RP11-208F18 - RP11-61M2 | chr1: 218446383-222066752 |
| | | (3.6 Mb) |
| 2 | RP11-1031M6 - RP11-61M2 | chr1: 215990775-221174779 |
| | | (5.2 Mb) |
| 3 | RP11-1148E24 - RP11-61M2 | chr1: 219486921-222066752 |
| | | (2.6 Mb) |
| 4 | RP11-1031M6 - RP11-61M2 | chr1: 214186825-222703737 |
| | | (8.5 Mb) |
| 5 | RP11-1031M6 - RP11-61M2 | chr1: 215521497-220657758 |
| | | (5.1 Mb) |
| 6 | RP11-1031M6 - RP11-61M2 | chr1: 212019895-221091768 |
| | | (9.07 Mb) |
| 7 | RP11-1031M6 - RP11-61M2 | ~5 Mb^{b} |
| Ref. 43 | NT | chr1: 217677120-227054747 |
| | | (10 Mb)^{c} |

^{a}based on NCBI build 35, hg17, May 2004;
^{b}Based on the publication by Kantarci et al. (Am. J. Med. Genet. A 2006; 140: 17-23);
^{c}Based on the publication by Slavotinek et al. (Eur. J. Hum. Genet. 2006; 14: 999-1008);
NT: not tested

In all cases, FISH (performed as described above) was used to confirm the presence of a heterozygous deletion of the region. Parental analyses in all families demonstrated two copies of the DISP1 locus; thus these deletions of 1q41q42 are de novo in all tested cases. All seven deletions were identified in our laboratory, although one case (subject 4) was previously published as a surviving Fryns syndrome patient with a normal karyotype (Van Hove J L, et al. Am J Med Genet 1995;59:334-340). The clinical features of another case (subject 7) were published following deletion identification (Kantarci S, et al. Am J Med Genet A 2006;140:17-23).

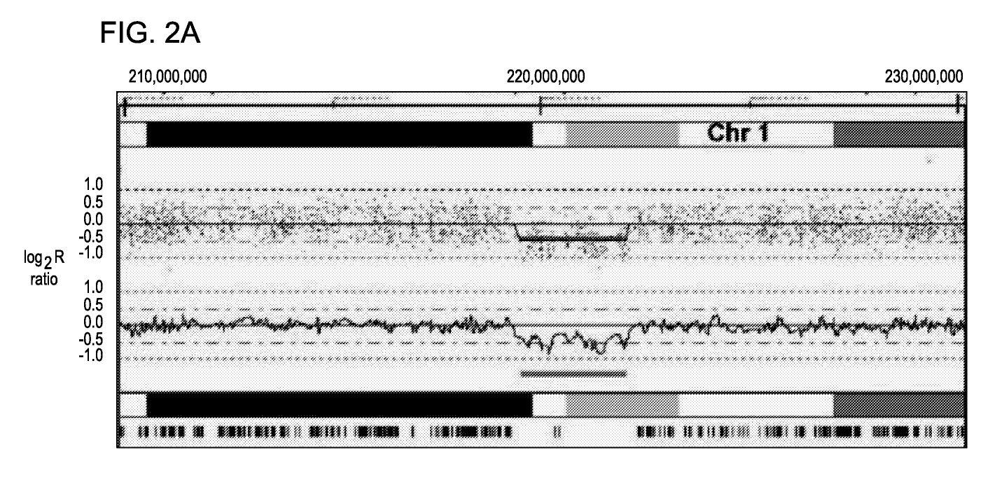

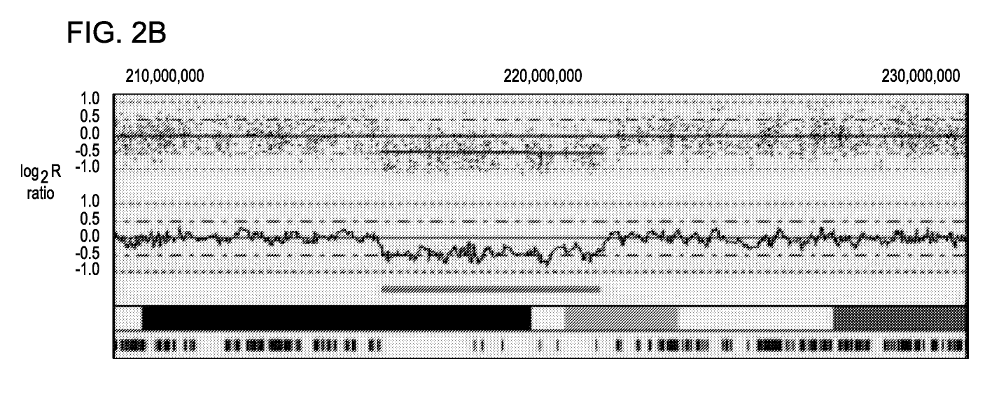

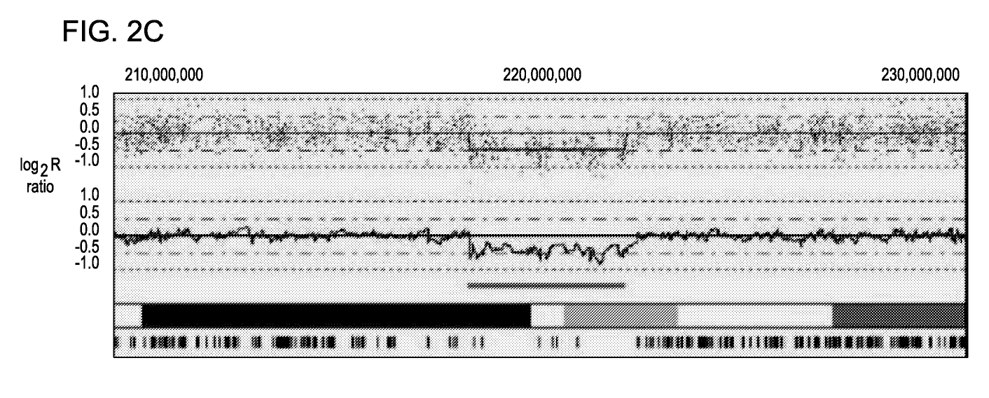

The 1q41q42 deletions were analyzed further by high-resolution microarray analysis using the Affymetrix GeneChip® 250K Sty chip (Affymetrix, Santa Clara, Calif., USA) as previously described (Ming J E, et al. Hum. Mutat. 27:467-473 (2006)) in order to (1) determine the full extent of the deletions at a higher resolution, and (2) define the smallest region of overlap (SRO) to identify candidate genes. Table 1 (above) and FIG. 2 show the results of this higher-resolution analysis. Subject 6 was found to have the largest deletion which is ˜9.07 Mb in size. The deletion in subject 3 was the smallest at 2.72 Mb. Subject 7 was not tested by high-resolution analysis.

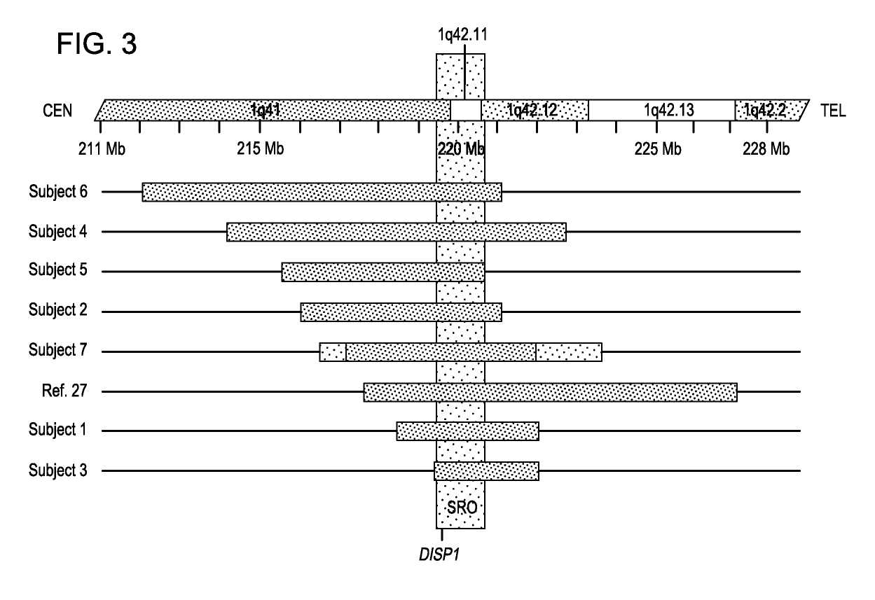

The SRO between the seven deletions is ˜1.17 Mb (Chr1:219486921-220657758) (FIG. 3). There are five genes within the SRO (Table 2), based on the University of California Santa Cruz known genes track (available on the Internet), and four of these five genes have known functions.

TABLE 2
Candidate genes identified in the smallest region
of overlap on 1q41q42.

| Gene | | |
|---|---|---|
| symbol | Description | Function |
| DISP1 | dispatched A | required in the SHH signalling |
| SUSD4 | sushi domain containing 4 | pathway unknown |
| CAPN2 | calpain 2, large subunit | calcium-regulated protease |
| TP53BP2 | tumor protein p53 binding | regulation of apoptosis |
| | protein, 2 | and cell growth |
| FBXO28 | F-box protein 28 | ubiquitination and degradation |

The common clinical features found in the seven subjects are shown in Table 3 below. Although none of the subjects have frank HPE, they show some common clinical features including significant developmental delay and distinct facial dysmorphism (frontal bossing, deep-set eyes, broad nasal tip, depressed nasal bridge, anteverted nares). Some subjects showed coarse facies in infancy, microcephaly, cleft palate, club feet, seizures, and short stature. Two subjects (subjects 4 and 7) had diaphragmatic hernia and lung hypoplasia and the clinical diagnosis of Fryns syndrome (Van Hove J L, et al. Am J Med Genet 1995;59:334-340; Kantarci S, et al. Am J Med Genet A 2006;140:17-23).

TABLE 3
Clinical features found in subjects with deletion of 1q41q42.

| | Subject | | | | | | |
|---|---|---|---|---|---|---|---|
| | 1 | 2 | 3 | 4^{a} | 5 | 6 | 7^{b} |
| Gender | F | M | M | M | M | M | F |
| Age at | 17 years | 20 mo. | 15 years | 16 years | 5 years, 7 mo. | 5 year, 10 mo. | birth |
| Diagnosis | | | | | | | |
| Facial Features | | | | | | | |
| Coarse facies | + | + | | + | − | + | + |
| Head | Micro- | Micro- | Dolicho- | | | Micro- | |
| | cephaly | cephaly | cephaly | | | cephaly, | |
| | | | | | | trigono- | |
| | | | | | | cephaly | |
| Hair | | | | | Recessed | Mildly | |
| | | | | | hairline | rough | |
| | | | | | with | texture | |
| | | | | | sparse | | |
| | | | | | eyebrows | | |
| Ears | | | | | Low set | Normal | Low set |
| Frontal bossing | | + | + | + | + | + | |
| Prominent brow | + | | + | | − | − | |
| Depressed nasal | + | + | + | + | − | + | + |
| bridge | | | | | | | |
| Broad nasal tip | + | | + | | − | + | + |
| Anteverted | + | | | + | − | + | + |
| nares | | | | | | | |
| Narrow | + | | + | | − | + | |
| bitemporal | | | | | | | |
| diameter | | | | | | | |
| Hypertelorism | | + | | | − | + | + |
| Palpebral | | Up slanting | | Down | Normal | Inner- | |
| fissures | | | | slanting | | canthal | |
| | | | | | | folds | |
| | | | | | | present | |
| Deep-set eyes | + | | + | | − | + | |
| Prominent | + | | + | | | | |
| mandible/ | | | | | | | |
| maxilla | | | | | | | |
| Mouth | | Thin upper | Short, | | Full lips, | Full, thick | Short |
| | | lip | upturned, | | prominent | lips, | philtrum, |
| | | | protruding | | philtrum | wide- | tented |
| | | | upper lip | | | spaced | upper lip |
| | | | | | | teeth | |
| Palate | High and | | | | Normal | Mildly | |
| | narrow | | | | | high | |
| Cleft palate | + | | | + | − | − | + |
| Trunk | Mild | | Accessory | Diaphragmatic | Normal | Accessory | Diaphragmatic |
| | pectus | | nipple, mild | hernia, | | nipple | hernia, |
| | excavatum | | scoliosis | lung | | | lung |
| | | | | hypoplasia | | | hypoplasia, |
| | | | | | | | VSD |
| Extremities | | Hip | | Contractures | | Mild limb | Mild |
| | | dysplasia | | | | shortening, | upper |
| | | | | | | hypermobile | arm |
| | | | | | | knees | shortening |
| Fingers | Slender, | | | Short, | | Mildly | |
| | mild | | | triangular, | | short | |
| | webbing | | | distal | | | |
| | between | | | phalanges | | | |
| | 3^{rd} and 4^{th} | | | | | | |
| Nails | | | | | | Hypoplastic | Hypoplasia |
| | | | | | | 5^{th} | |
| | | | | | | toenails | |
| Feet | Wide | Bilateral | | Bilateral | | Pes plano- | Bilateral |
| | space | club feet | | club feet | | valgus, | club feet |
| | between | | | | | wide | |
| | first and | | | | | space | |
| | second | | | | | between | |
| | toes, high | | | | | first and | |
| | arches | | | | | second | |
| | | | | | | toes | |
| Neurological | Seizures, | Seizures, | Seizures, | Seizures, | Seizures, | Seizures, | |
| | normal | normal | Enlarged | abnormal | abnormal | abnormal | |
| | brain MRI | EEG, | ventricles | EEG, | EEG, | EEG, | |
| | | normal | by MRI | small | normal | normal | |
| | | brain MRI | | cerebellum | MRI | MRI, | |
| | | | | by CT | | enlarged | |
| | | | | scan, | | ventricles | |
| | | | | gyral | | prenatally | |
| | | | | malformations | | | |
| | | | | by | | | |
| | | | | MRI | | | |
| Measurements | | | | | | | |
| at last | | | | | | | |
| evaluation | | | | | | | |
| Height | 3^{rd} %ile | <5^{th} %ile | 25^{th}-50^{th} | | 4^{th} %ile | <3^{th} %ile | |
| | | | %ile | | | | |
| Weight | 3^{rd} %ile | <5^{th} %ile | 10^{th} %ile | | 14^{th} %ile | <3^{rd} %ile | |
| FOC | 2^{nd} %ile | <2^{nd} %ile | 50^{th} %ile | | 20^{th} %ile | <5^{th} %ile | |

^{a}Based on the publication by Van Hove et al. Ibid;
^{b}Based on the publication by Kantarci et al. Ibid; + = feature present, − = feature not present, blank = subject was not examined for that feature.

### EXAMPLE 2

### Identification of Novel Genetic Disorders Associated with Chromosomal Abnormalities at 16p11.2-p.12.2

### a) Identification and Characterization of Pericentromeric Imbalances by Array CGH

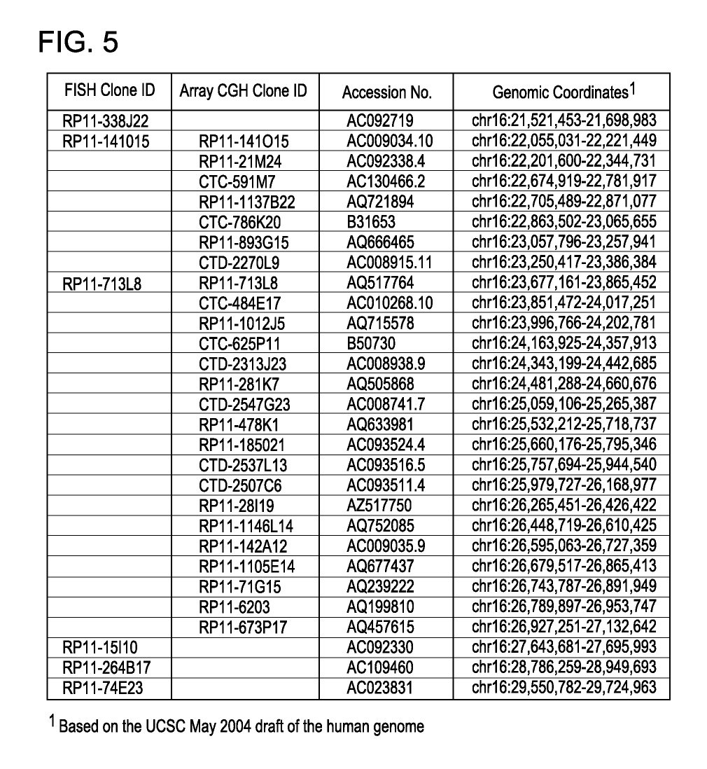

Of over 8,000 patients initially screened using the SignatureChip® targeted microarray, which includes a minimum of 3-6 overlapping BAC clones at the most proximal end of the pericentromeric region for each chromosome arm (excluding the short arms of the acrocentric chromosomes), 26 were found to have a pericentromeric imbalance. All 26 patients were originally referred by physicians for testing. The most common clinical presentations of the patients were mental retardation, developmental delay, or multiple congenital anomalies. Twelve patients had previous normal cytogenetic analysis, subtelomere FISH, and/or locus-specific FISH. The 26 patients in whom the targeted microarray identified a pericentromeric deletion or duplication were selected for further characterization by a higher-density pericentromeric array which includes contigs of clones placed ˜0.5 Mb apart that span ˜5 Mb of the most proximal unique sequence adjacent to the centromere on all 43 unique pericentromeric regions (Ballif, B. C. et al. Development of a high-density pericentromeric region BAC clone set for the detection and characterization of small supernumerary marker chromosomes by array CGH. Genet Med 2007 Mar;9(3):150-62. FIG. 5 shows the genomic coordinates of BAC clones used in pericentromeric array CGH and FISH to delineate breakpoints in patients with abnormalities of 16p11.2p12.2.

### b) Discovery of a Novel Microdeletion Syndrome on 16p11.2p12.2

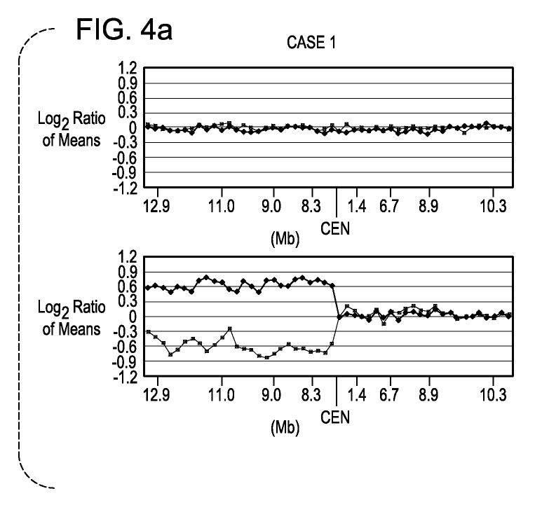

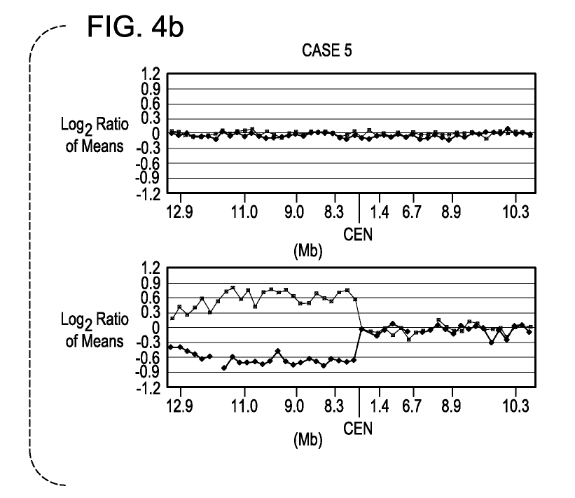

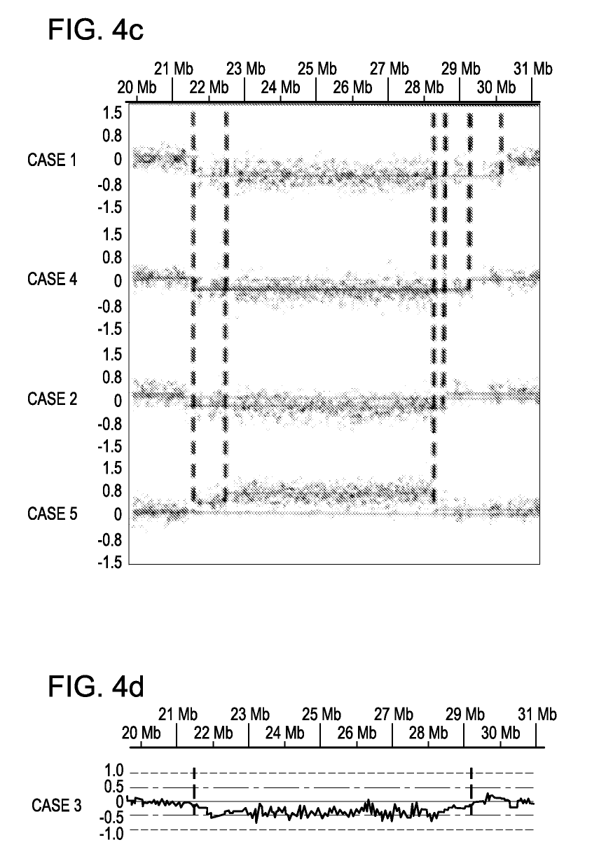

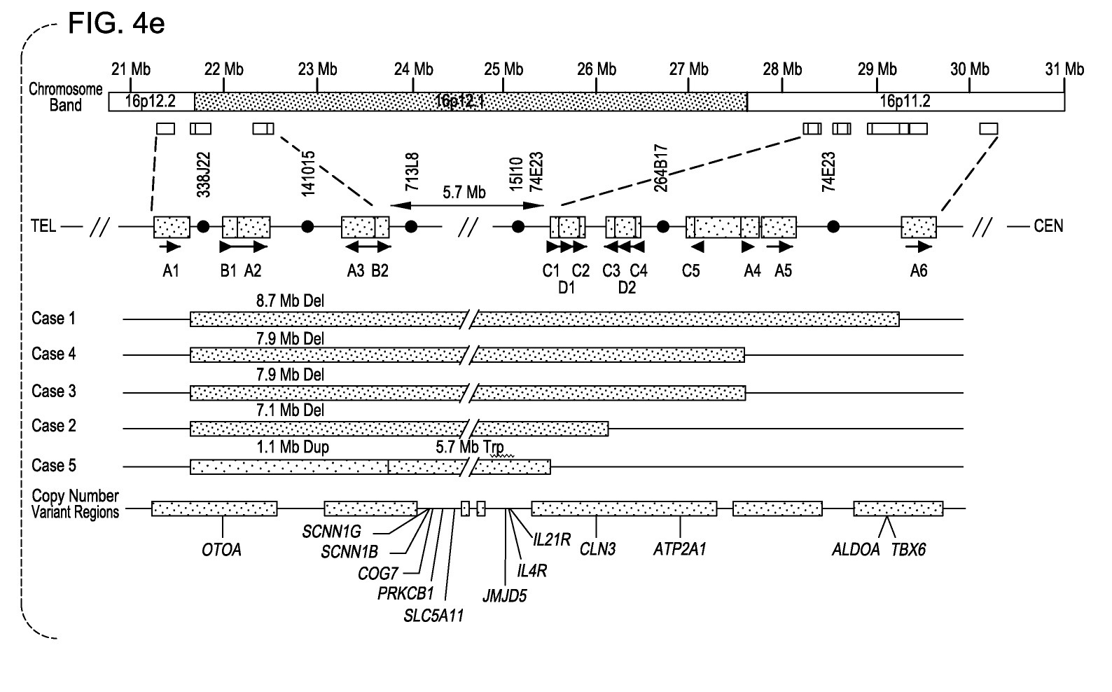

Of the 26 cases characterized by the pericentromeric microarray, four had recurrent deletions of 16p11.2p12.2 (cases 1, 2, 3 and 4) (FIG. 4). Parental analyses in three of these cases (1, 3 and 4) demonstrated that these are de novo chromosome abnormalities. To clarify the sizes of the deletions further, we analyzed all four cases with a high-density BAC-based microarray spanning approx. 5 Mb of the most proximal unique sequence adjacent to the centromere for all 43 unique percentromeric regions of the human genome (shown in FIGS. 4A and B for cases 1 and 5, respectively). Because all four of these abnormalities extended beyond the ˜5 Mb coverage of the pericentromeric microarray, the full extent of each abnormality was characterized using (i) whole-genome tiling-path array CGH with a median probe spacing of 6 kb (for cases 1, 2 and 4; NimbleGen Systems, Inc., Madison, Wis.; Selzer, R. R. et al. Genes Chromosomes Cancer 44, 305-19 (2005)) and/or Affymetrix 250K SNP arrays (for cases 3 and 4; Affymetrix, Santa Clara, Calif.; Ming, J. E. et al. Hum Mutat 27, 467-73 (2006)). The results are shown in FIGS. 4C and D. FISH analysis using BAC clones that map to the various breakpoint regions and previously published methodology (Shaffer, L. G. et al. Am J Hum Genet 55, 968-74 (1994); Shaffer, L. G., et al. Am J Med Genet 69, 325-31 (1997)) confirmed the results of the whole-genome arrays. All four deletions of 16p11.2p12.2 share the same distal breakpoint located ˜21.4 Mb from the 16p telomere. However, the proximal breakpoints were ˜28.5 Mb (case 2), ˜29.3 Mb (cases 3 and 4) and ˜30.1 Mb (case 1) from the 16p telomere, resulting in overall deletion sizes of ˜7.1 Mb, ˜7.9 Mb and ˜8.7 Mb, respectively.

Sequence analysis of the 16p11.2p12.2 region using the University of California Santa Cruz genome browser and the Human Genome Segmental Duplication Database identified a complex arrangement of segmental duplications, some of which directly flank the deletion breakpoints (FIG. 4E). These segmental duplications were arbitrarily assigned the alphanumeric identifications of A1-A6, B1-B2, C1-C5 and D1-D2 for simplicity in describing these regions and the particular rearrangements associated with them. Segmental duplications A1-A6 are 71-146 kb in size and share >98% sequence identity. Segmental duplication A1 is located at the distal breakpoint in all four deletion cases and is in the same orientation as A2, A4 and A5 (tandemly repeated), and A6. The proximal breakpoints in cases 4 and 3 are located just distal to the tandemly repeated segmental duplications A4 and A5, whereas the proximal breakpoint in case 1 is located just distal to A6. The B1-B2 segmental duplications are ˜35 kb in size, are in the same orientation, and share >99% sequence identity. Segmental duplications C1-C5 are part of a more complex cluster that includes D1-D2. C1-C5 are 19-59 kb in size and share >99% sequence identity. The proximal breakpoint of case 2 is located just distal to C3. D1-D2 are ˜133 kb in size, share >99% sequence identity, and encompass the flanking segmental duplications C1-C2 and C3-C4, respectively. The locations of the breakpoints in these four cases with respect to the location and orientation of the segmental duplications in the 16p11.2-12.2 region suggest that nonallelic homologous recombination mediated these rearrangements.

The clinical features of the four patients with microdeletions of 16p11.2p12.2 are listed in Table 4 below and consist of distinct facial features, including flat facies, down-slanting palpebral fissures, low-set and malformed ears, and eye anomalies. Other commonly described features include orofacial clefting, heart defects, frequent ear infections with potential hearing loss, short stature, minor hand and foot anomalies, feeding difficulties, hypotonia, and cognitive and developmental delays.

TABLE 4
Clinical findings in patients with copy-number imbalances of 16p11.2p12.2.

| | | | | | trp(16)(p11.2p12.1) |
|---|---|---|---|---|---|
| Imbalance | del(16)(p11.2p12.2) | | | | dup(16)(p12.1p12.2) |
| Case no. | 1 | 4 | 3 | 2 | 5 |
| Gender | Female | Female | Female | Female | Female |
| Age at | 13 yr 8 mo | 2 yr 9 mo | 3 yr 1 mo | 13 yr 9 mo | 10 yr 11 mo |
| diagnosis | | | | | |
| Size of | 8.7 Mb deletion | 7.8 Mb deletion | 7.8 Mb deletion | 7.1 Mb deletion | 5.7 Mb |
| imbalance | | | | | triplication; |
| | | | | | 1.1 Mb |
| | | | | | duplication |
| Previous | Multiple normal | Normal | Normal | Normal | Normal |
| cytogenetic | karyotypes; normal | karyotype; | karyotype; | karyotype | karyotype; |
| testing | subtelomere FISH, | normal | normal fragile X | | normal 7q11.23 |
| | 22q11.2 FISH, | subtelomere | testing | | FISH and |
| | SNRPN | FISH, 22q11.2 | | | fragile X testing |
| | methylation, Rett, | and 17p13.3 | | | |
| | Noonan, and | FISH, and | | | |
| | CHARGE | SNRPN | | | |
| | syndrome testing | methylation | | | |
| | | testing | | | |
| Cranio- | Head | Head | Head | Head | Head |
| facial | circumference 10^{th} | circumference 8^{th} | circumference | circumference | circumference (−1SD); |
| | percentile; long, | percentile; frontal | 25^{th} percentile; | 25^{th} percentile; | round |
| | narrow and flat | bossing | flat facies; frontal | flat facies | face with full |
| | facies; high | | bossing | | cheeks |
| | forehead | | | | |
| Mouth & | Pierre Robin | | Prominent jaw; | Micrognathia | High-arched |
| Jaw | Sequence (cleft lip | | mouth open with | | palate; some |
| | and palate, | | drooling | | retrognathia; |
| | glossoptosis, | | | | wide mouth; |
| | micrognathia); | | | | long philtrum; |
| | mouth open with | | | | thin upper lip |
| | drooling | | | | |
| Eyes | Downslanting | Downslanting | Downslanting | Short and | Narrow and |
| | palpebral fissures; | palpebral fissures; | palpebral fissures; | downslanting | slightly short |
| | bilateral epicanthal | mild epicanthal | left epicanthal | palpebral | palpebral |
| | folds; deep-set | folds; deep-set | fold; | fissures; relative | fissures; relative |
| | eyes; absent tear | eyes | hypotelorism | hypotelorism | hypertelorism (0 |
| | ducts; strabismus | | | (3^{rd}-10^{th} | to +1SD); ptosis |
| | | | | percentile) | (right eye); |
| | | | | | strabismus (left |
| | | | | | eye); hyperopia |
| Ears | Low-set and | Posteriorly | Posteriorly | Frequent ear | Low-set and |
| | malformed ears; | rotated ears; | rotated ears; | infections | posteriorly |
| | frequent ear | prominent | lacking lobes; | required PE | rotated ears; |
| | infections required | antihelix; | frequent ear | tube placement | hyperacusis |
| | PE tube placement; | frequent ear | infections | | |
| | cholesteatoma; | infections | required PE tube | | |
| | moderate hearing | required PE tube | placement | | |
| | loss | placement | | | |
| Nose | Wide nasal bridge; | Upturned nose | Short, prominent | | Short nose with |
| | anteverted nares; | | nose; slightly | | wide nasal |
| | chronic sinusitis | | bulbous and bifid | | bridge; round |
| | | | | | nasal tip; |
| | | | | | anteverted nares |
| Cardio- | Cyanosis; syncope | Tricuspid | Normal | | Persistent |
| vascular | episodes; | regurgitation | echocardiogram | | tachycardia |
| | bradycardia | (Epstein's | | | |
| | | anomaly); | | | |
| | | pulmonary | | | |
| | | stenosis | | | |
| Skeletal & | Height 3^{rd} | Height 25^{th} | Height 25^{th}-50^{th} | Height <3^{rd} | Height −3 to −4SD; |
| Muscular | percentile; weight | percentile; weight | percentile; weight | percentile, | weight −2SD; |
| | 3^{rd} percentile; | 3^{rd} percentile; | 50^{th} percentile; | weight 10^{th}-25^{th} | hypotonia; |
| | hypotonia; short | hypotonia; arched | hypotonia; single | 25^{th} percentile; | bridged palmar |
| | fingers and hands; | dermatoglyphics; | palmar crease | single palmar | creases (left |
| | single palmar | minimal | (right hand) | crease (left | hand); short |
| | crease (right hand); | syndactyly of toes | | hand); | fifth fingers; |
| | proximally placed | (2, 3 - left foot | | camptodactyly; | prominent |
| | thumbs; | only) | | absent flexion | finger tip pads; |
| | camptodactyly; | | | creases | bilateral hallux |
| | absent flexion | | | | valgus, minimal |
| | creases; mild | | | | 2, 3 toe |
| | cutaneous | | | | syndactyly |
| | syndactyly; | | | | |
| | unsteady gate | | | | |
| Gastr- | Feeding difficulties | Feeding | Feeding | Feeding | Feeding |
| intestinal & | in infancy; GE | difficulties in | difficulties in | difficulties in | difficulties in |
| Nutrition | reflux; uses G-tube | infancy; GE | infancy; GE | infancy; GE | infancy |
| | | reflux | reflux | reflux | |
| Psycho- | Significant delay; | Significant delay; | Significant delay; | Significant | Significant |
| motor & | speaks very few | no speech; uses a | starting to put two | delay; mild to | delay; IQ of 42 |
| Cognitive | words; uses sign | limited number of | to three words | moderate mental | (WISC-IV |
| Delay | language of ~500 | signs; no self- | together; uses | retardation (IQ | assessment at |
| | words; limited self- | help skills | some signs | reportedly in | age 9); 12^{th} |
| | help skills | | | 50's); can trace | percentile for |
| | | | | name and count | reading, <1^{st} |
| | | | | to 12; has some | percentile for |
| | | | | self-help skills | spelling and |
| | | | | | math (WRAT-3 |
| | | | | | assessment at |
| | | | | | age 9) |
| Behavioral | | Irritability; head | | Anxiety; | Friendly and |
| | | banging; hand | | energetic | talkative; |
| | | flapping | | | ADHD; anxiety |
| | | | | | and nervousness |
| | | | | | leading to nail |
| | | | | | biting and skin |
| | | | | | picking to the |
| | | | | | point of trauma |
| Other | Sleep apnea; | Wide-based | | Sleep problems; | Growth |
| | staring spells; | nipples; café-au- | | potential | hormone |
| | potential | lait macule on | | insensitivity to | deficiency |
| | insensitivity to | chest | | pain; café-au- | |
| | pain; chronic lung | | | lait macules on | |
| | disease; significant | | | chest, right arm, | |
| | hair growth on | | | and right leg | |
| | back and legs | | | | |

One hundred and four known genes are located within the 8.7 Mb region defined by the largest of the 16p11.2p12.2 deletions identified in this study. Mutations in at least seven genes located within the largest deletion region are known to be associated with disease phenotypes, and at least six additional genes have known functions or belong to gene families whose functions would make them promising candidate genes for the phenotypic features of microdeletions of 16p11.2p12.2. These 13 genes, together with their functions and associations with known syndromes are listed in Table 5.

TABLE 5
Candidate genes in 16p11.2p12.2 and their functions (where known).

| | OMIM | Disease Associations (if | | |
|---|---|---|---|---|
| Gene | No. | known) | Function (if known) | References |
| OTOA | 607038 | Autosomal recessive deafness | Inner ear protein restricted to the interface | 1 |
| | | | between the apical surface of sensory | |
| | | | epithelia and their overlying acellular gels | |
| SCNN1G | 600761 | Autosomal dominant Liddle | Structural subunit of the amiloride- | 2, 3 |
| | | syndrome (human | sensitive epithelial sodium channel | |
| | | hypertension); may also play a | expressed in the distal nephron; | |
| | | role in cystic-fibrosis-like lung | constitutive activation or overexpression | |
| | | disease | results in Liddle syndrome | |
| SCNN1B | 600760 | Autosomal dominant Liddle | Structural subunit of the amiloride- | 4, 5 |
| | | syndrome (human | sensitive epithelial sodium channel | |
| | | hypertension); homozygous | expressed in the distal nephron; | |
| | | loss of function results in | constitutive activation or overexpression | |
| | | pseudohypoaldosteronism type | results in Liddle syndrome; homozygous | |
| | | I (opposite phenotype) | loss of function results in | |
| | | | pseudohypoaldosteronism type I | |
| COG7 | 606978 | Autosomal recessive congenital | Component of the conserved oligomeric | 6, 7 |
| | | disorder of glycosylation type | Golgi (COG) complex involved in | |
| | | IIe | intracellular transport and glycoprotein | |
| | | | modification | |
| PRKCB1 | 176970 | Homozygous knockout mice | Member of the protein kinase C gene | 8, 9 |
| | | develop immunodeficiency | family; Mediates prevention of mouse | |
| | | | neural tube defects by inositol | |
| SLC5A11 | 610238 | Candidate gene for seizure | Member of the sodium/glucose | 10 |
| | | phenotype | cotransporter gene family | |
| JMJD5 | NA | Unknown | Novel member of jumonji gene family; | 11 |
| | | | jumonji gene family members function in | |
| | | | transcriptional repression and/or chromatin | |
| | | | regulation during development of the heart | |
| | | | and liver, neural tube fusion, and | |
| | | | hematopoeisis in mice | |
| IL4R | 147781 | Specific haplotypes associated | Component of the interleukin 4 receptor | 12 |
| | | with atopy | which plays a major role in interleukin-4 | |
| | | | signal cascade to produce immunoglobulin E | |
| IL21R | 605383 | Knockout mice suggest IL21R | Type I cytokine receptor for hematopoietic | 13 |
| | | plays a role in the transition | growth factors and interleukins | |
| | | from innate to adaptive | | |
| | | immunity | | |
| CLN3 | 607042 | Autosomal recessive Batten | Unknown | 14 |
| | | disease (severe | | |
| | | neurodegeneration) | | |
| ATP2A1 | 108730 | Autosomal recessive Brody | Calcium-transporting ATPase | 15 |
| | | myopathy (disorder of skeletal | | |
| | | muscle function) | | |
| ALDOA | 103850 | Autosomal recessive aldolase | Glycolytic enzyme; Sole adolase isozyme | 16, 17 |
| | | deficiency resulting in | expressed during embryonic development | |
| | | hemolytic anemia and possibly | and one of two isozymes expressed in adult | |
| | | mental retardation and | brain and nervous tissue | |
| | | dysmorphic features | | |
| TBX6 | 602427 | Knockout mice develop | Transcription factor that specifies paraxial | 18 |
| | | paraxial neural tube-like | mesoderm formation | |
| | | structures | | |

1. Zwaenepoel, I. et al. Proc Natl Acad Sci USA 99, 6240-5 (2002).
2. Hansson, J. H. et al. Nat Genet 11, 76-82 (1995).
3. Mall, M. et al. Nat Med 10, 487-93 (2004).
4. Hansson, J. H. et al. Proc Natl Acad Sci USA 92, 11495-9 (1995).
5. Chang, S. S. et al. Nat Genet 12, 248-53 (1996).
6. Ungar, D. et al. J Cell Biol 157, 405-15 (2002).
7. Wu, X. et al. Nat Med 10, 518-23 (2004).
8. Leitges, M. et al. Science 273, 788-91 (1996).
9. Cogram, P. et al. Hum Mol Genet 13, 7-14 (2004).
10. Roll, P. et al. Gene 285, 141-8 (2002).
11. Takeuchi, T. et al. Dev Dyn 235, 2449-59 (2006).
12. Ober, C. et al. Am J Hum Genet 66, 517-26 (2000).
13. Kasaian, M. T. et al. Immunity 16, 559-69 (2002).
14. Phillips, S. N. J Neurosci Res 79, 573-83 (2005).
15. Odermatt, A. et al. Nat Genet 14, 191-4 (1996).
16. Beutler, E. et al. Trans Assoc Am Physicians 86, 154-66 (1973).
17. Hurst, J. A. et al. Am J Med Genet 28, 965-70 (1987).
18. Chapman, D. L. & Papaioannou, V. E. Nature 391, 695-7 (1998).

c) Characterization of a Complex de novo Duplication/Triplication of 16p11.2p12.2

A complex de novo pericentromeric abnormality (case 5) involving a duplication and triplication of the same region of 16p11.2p12.2 that is deleted in the four microdeletion cases described above was also identified. Because this abnormality extended beyond the ˜5 Mb coverage of the pericentromeric microarray, whole-genome oligonucleotide array CGH (NimbleGen™) was performed to refine the breakpoints further (FIGS. 4B, C and E). By this analysis, the distal duplication was determined to be ˜1.1 Mb in size with the same distal breakpoint at segmental duplication A1 as all four 16p11.2p12.2 microdeletion cases. The proximal duplication breakpoint is the same as the distal triplication breakpoint and is located just proximally to segmental duplication B2. The triplicated segment is ˜5.7 Mb in size and overlaps the SRO of the four 16p11.2p12.2 microdeletion cases with a proximal breakpoint just distal to segmental duplication C1. These duplication and triplication breakpoints were confirmed by FISH using BAC clones mapping within these regions of 16p11.2p12.2.

The clinical features of case 5 are listed in Table 4 above. Briefly, the facial findings include round face, broad/flat nasal bridge, anteverted nares, long philtrum, thin upper lip, narrow palpebral fissures, low-set ears, and eye anomalies. Other features include intact but high arched palate; hypotonia; mild hand and foot anomalies; and behavioral and neurological abnormalities including staring spells, attention deficit hyperactivity disorder (ADHD), anxiety, and hyperacusis.

While the present invention has been described with reference to specific embodiments thereof, it should be understood by those skilled in the art that various changes may be made and equivalents may be substituted without departing from the true spirit and scope of the invention. In addition, many modifications may be made to adapt a particular situation, material, composition of matter, method, method step or steps, for use in practicing the present invention. All such modifications are intended to be within the scope of the claims.

All of the publications, patent applications, and patents cited in this application are herein incorporated by reference in their entirety to the same extent as if each individual publication, patent application or patent was specifically and individually indicated to be incorporated by reference in its entirety.

We claim:

1. A method for detecting the presence of a genetic disorder in a patient, comprising:
(a) providing a DNA-containing test sample from the patient; and
(b) identifying in the test sample the deletion or amplification of target chromosome loci selected from the group consisting of: 1q41q42 and 16p11.2p12.2,
wherein the deletion or amplification of the target chromosome loci is indicative of the presence of the genetic disorder in the patient, and wherein the genetic disorder is not Fryns syndrome or congenital diaphragmatic hernia (CDH).

2. The method of claim 1, wherein the deletion or amplification of the target chromosome loci is detected by means of microarray comparative genomic hybridization, SNP microarray, fluorescence in situ hybridization, restriction fragment length polymorphism, polymerase chain reaction, Southern blotting, pulse field gel electrophoresis, multiplex ligation dependent probe amplification, or real time PCR.

3. The method of claim 1, wherein step (b) comprises:
(i) labeling nucleic acids from the test sample with a first detectable label to provide labeled test nucleic acids;
(ii) labeling nucleic acids from a control sample with a second, different, detectable label to provide labeled control nucleic acids;
(iii) contacting the labeled test nucleic acids and the labeled control nucleic acids with a plurality of target nucleic acids specific for chromosomal loci selected from the group consisting of: 1q41a42 and 16p11.2p12.2; and
(iv) comparing intensities of signals from labeled test nucleic acids hybridized to each target nucleic acid with intensities of signals from labeled control nucleic acids hybridized to each target nucleic acid.

4. The method of claim 3, wherein at least one of the first and second detectable labels is a fluorescent molecule.

5. The method of claim 3, wherein the plurality of target nucleic acids are provided on a solid surface.

6. The method of claim 3, wherein the plurality of target nucleic acids are capable of specifically hybridizing to target regions of the chromosomal loci.

7. The method of claim 1, wherein the test sample is selected from the group consisting of: blood, serum, urine, saliva, amniotic fluid, chorionic villus, semen and skin.

8. The method of claim 1 wherein the genetic disorder is a microdeletion syndrome that correlates with a deletion of 1q41q42.

9. The method of claim 8, wherein the genetic disorder is characterized by the presence of at least one for the following phenotypes: developmental delay, facial dysmorphism, coarse facies in infancy, microcephaly, cleft palate, clubfeet, seizures, short stature, diaphragmatic hernia, and lung hypoplasia.

10. The method of claim 1, wherein the genetic disorder is a microdeletion syndrome that correlates with a deletion of 16p11.2p12.2.

11. The method of claim 10, wherein the genetic disorder is characterized by the presence of at least one for the following phenotypes: developmental delay, facial dysmorphism, Pierre Robin Sequence, downslanting palpebral fissures, bilateral epicanthal folds, deep-set eyes, absent tear ducts, strabismus, low-set and malformed ears, short and prominent nose, and small stature.

12. The method of claim 1, wherein the genetic disorder correlates with an amplification of 16p11.2p12.2.

13. The method of claim 12, wherein the genetic disorder correlates with a duplication or a triplication of 16p11.2p12.2.

14. A microarray comprising a plurality of target nucleic acid probes that specifically hybridize to a region of a chromosomal locus selected from the group consisting of: 1q41q42 and 16p11.2p12.2.

15. A kit for use in the detection of a genetic disorder comprising:
(a) a container; and
(b) at least one nucleic acid probe that specifically hybridizes to a region of a target chromosome loci selected from the group consisting of: 1q41q42 and 16p11.2p12.2.

16. A kit for use in the detection of a genetic disorder comprising:
(a) a container; and
(b) at least one primer pair that is capable of specifically amplifying a region of a target chromosome loci selected from the group consisting of: 1q41q42 and 16p11.2p12.2.
